(19) United States
(12) Patent Application Publication
(10) Pub. No.: US 2024/0261354 A1
(43) Pub. Date: Aug. 8, 2024

(54) METHODS FOR IMPROVING BONE QUALITY AND PROMOTING CARTILAGE DIFFERENTIATION BY CHRYSOPHYLLUM CAINITO EXTRACT AND MANUFACTURING METHOD THEREOF

(71) Applicant: TCI CO., LTD., Taipei (TW)

(72) Inventors: YUNG-HSIANG LIN, Taipei (TW); HSIAO-NAI LIN, Taipei (TW)

(21) Appl. No.: 18/395,764

(22) Filed: Dec. 26, 2023

Related U.S. Application Data
(60) Provisional application No. 63/483,304, filed on Feb. 6, 2023.

(51) Int. Cl. A61K 36/185; A61K 31/366; A61P 19/00; A61P 19/04

(52) U.S. Cl. CPC A61K 36/185; A61K 31/366; A61P 19/00; A61P 19/04; A61K 2236/19; A61K 2236/30

(57) ABSTRACT

Methods for improving bone quality and promoting cartilage differentiation in a subject in need thereof are provided. The methods include: administering to the subject an effective amount of Chrysophyllum cainito extract. The Chrysophyllum cainito extract is obtained by extracting early-harvested fruit of Chrysophyllum cainito by a cellulase complex solution. The cellulase complex solution is prepared by adding 0.1% of cellulase complex in water. The Chrysophyllum cainito extract has at least one of the following effects of: promoting to production of osteocalcin by osteoblasts, promoting osteocalcin content in blood, improving bone density of lumbar vertebra, and promoting secretion of glycosaminoglycans by chondrocytes.

### CROSS-REFERENCE TO RELATED APPLICATION

This application claims the benefit of U.S. provisional application Ser. No. 63/483,304, filed on Feb. 6, 2023. The entirety of the above-mentioned patent application is hereby incorporated by reference herein and made a part of the specification.

### BACKGROUND

### Technical Field

The present disclosure relates to application of a Chrysophyllum cainito extract, and particularly relates to a use method of a Chrysophyllum cainito extract. The Chrysophyllum cainito extract has the effects of improving bone quality or promoting cartilage differentiation and the like.

### Related Art

It is known that bone density is a dynamic equilibrium state. Osteophages will decompose bone and release calcium into the blood, also metabolize old bone, while osteoblasts are responsible for generating new bone. However, the decrease of hormones after middle age also leads to the slowdown of osteogenesis and the increase of phagocytosis to increase, which may easily cause osteoporosis.

Chrysophyllum cainito belongs to the perennial tree of the family Lymphaceae, native to in central Panama. The adult fruit tree is about 10 meters tall, and its characteristic is that the back of the leaves has golden fuzz. The appearance and size of the Chrysophyllum cainito are similar to apples. In the cross section of the fruit, star-shaped white flesh can be seen radiating outward from the core, so it is also commonly known as a star apple. There are two types of skin: purple skin and green skin when ripening. The purple skin variety has a thicker skin, and the flesh changes from purple to white. The green skin variety has a thin skin, and the flesh is white.

### SUMMARY

In view of this, in order to further seek for a wider new application of Chrysophyllum cainito, the present disclosure provides a composition for applying a Chrysophyllum cainito extract to manufacture and for improving bone quality and promoting cartilage differentiation.

In some embodiments, a method for improving bone quality of a subject in need thereof, comprising: administering to the subject an effective amount of Chrysophyllum cainito extract. The Chrysophyllum cainito extract is obtained by extracting early-harvested fruit of Chrysophyllum cainito by a cellulase complex solution, and the cellulase complex solution is prepared by adding 0.1% of cellulase complex in water.

In some embodiments, a weight ratio of the cellulose complex solution to the early-harvested fruits of Chrysophyllum cainito is 1:(5-10).

In some embodiments, the extraction of early-harvested fruits of Chrysophyllum cainito includes steps of: ice crystal wall breaking, low-temperature extraction, and high-temperature extraction. The ice crystal wall breaking step is performed by letting the early-harvested fruits stand at 0-10° C. for 7 days, the low-temperature extraction step is performed at 45-60° C. for 50-70 min, and the high-temperature extraction step is performed at 85±5° C. for 50-70 min.

In some embodiments, the Chrysophyllum cainito extract promotes production of osteocalcin by osteoblasts of the subject to achieve the effect of improving bone quality.

In some embodiments, the Chrysophyllum cainito extract promotes osteocalcin content in blood of the subject to achieve the effect of improving bone quality.

In some embodiments, the Chrysophyllum cainito extract improves the bone density of lumbar vertebra of the subject to achieve the effect of improving bone quality.

In some embodiments, a method for promoting cartilage differentiation in a subject in need thereof, comprising: administering to the subject an effective amount of Chrysophyllum cainito extract. The Chrysophyllum cainito extract is obtained by extracting early-harvested fruit of Chrysophyllum cainito by a cellulase complex solution, and the cellulase complex solution is prepared by adding 0.1% of cellulase complex in water.

In some embodiments, the Chrysophyllum cainito extract promotes secretion of glycosaminoglycans (GAGs) by chondrocytes in the subject to achieve the effect of promoting cartilage differentiation.

In some embodiments, a manufacturing method of Chrysophyllum cainito extract, comprising a step of: extracting early-harvested fruit of Chrysophyllum cainito by a cellulase complex solution prepared by adding 0.1% of cellulase complex in water, wherein the extraction comprises steps of: ice crystal wall breaking, low-temperature extraction, and high-temperature extraction. The ice crystal wall breaking step is performed by letting the early-harvested fruit stand at −10° C. to 0° C. for 7 days, the low-temperature extraction is performed at 45-60° C. for 50-70 min, and the high-temperature extraction is performed at 85±5° C. for 50-70 min.

In some embodiments, the Chrysophyllum cainito extract includes at least 30 ppm of myricetin.

In conclusion, the Chrysophyllum cainito extract in any embodiment can be used for manufacturing the composition for improving bone quality and promoting cartilage differentiation. In some embodiments, the Chrysophyllum cainito extract has at least one of the following effects of: promoting production of osteocalcin by osteoblasts, promoting osteocalcin content in blood, improving bone density of the lumbar vertebra, or promoting secretion of glycosaminoglycan by chondrocytes.

### BRIEF DESCRIPTION OF THE DRAWINGS

FIG. 1 is a bar chart showing the relative osteocalcin content.

FIG. 2 is images showing the result of a glycosaminoglycan test.

FIG. 3 is a bar chart showing the osteocalcin content in blood of human subjects.

FIG. 4 is a bar chart showing the osteocalcin content in blood of other human subjects.

FIG. 5 is a line chart showing the T-score of human subjects.

FIG. 6 is a line chart showing the T-score of other human subjects.

FIG. 7 is an HPLC graph of a Chrysophyllum cainito extract.

FIG. 8 is a bar chart showing the relative osteocalcin content.

### DETAILED DESCRIPTION

The term “extract” used herein refers to a product manufactured by extraction. Herein, the extract may be present in the form of a solution dissolved in a solvent, or in the form of a concentrate or essence that is free or substantially free of solvent.

The term “receptor” described herein refers to human or non-human mammals, preferably humans.

As used herein, the “Chrysophyllum cainito” refers to an intact fruit of a Chrysophyllum cainito plant, and is also commonly referred to as star apple. In some embodiments, the Chrysophyllum cainito is an early-harvested fruit of the purple skin variety, and the early-harvested fruit refers to a fruit in the green appearance period. In some embodiments, the intact fruit includes pericarp, flesh and seeds.

In some embodiments, the Chrysophyllum cainito may be fresh, dried, or frozen. In some embodiments, the drying may be air-drying, sun-drying, shade-drying, or freeze-drying. In some embodiments, the Chrysophyllum cainito may further include intact, chopped, diced, milled, ground, or otherwise processed fruit to affect the size and entity integrity of the raw material. In some embodiments, the Chrysophyllum cainito is produced in Taiwan, China.

In some embodiments, a method for improving bone quality of a subject in need thereof, comprising: administering to the subject an effective amount of Chrysophyllum cainito extract. The Chrysophyllum cainito extract is obtained by extracting early-harvested fruit of Chrysophyllum cainito by a cellulase complex solution, and the cellulase complex solution is prepared by adding 0.1% of cellulase complex in water. In some embodiments, the cellulase complex is the generic term of an enzyme that degrades cellulase or glucoside bonds. In some embodiments, the cellulase complex includes β-glucanase. Herein, the β-glucanase is an endo-β-glucanase that hydrolyzes (1,3)- or (1,4)-bonds in β-D-glucan. Herein, the extraction is to soak the Chrysophyllum cainito in water for a period of time.

In some embodiments, a weight ratio of the cellulose complex solution to the early-harvested fruits of Chrysophyllum cainito is (5-10):1.

In some embodiments, the extraction of early-harvested fruits of Chrysophyllum cainito includes steps of: ice crystal wall breaking, low-temperature extraction and high-temperature extraction. In some embodiments, the ice crystal wall breaking step is performed by letting the early-harvested fruits stand at 0-10° C. for 7 days, to break cell walls so as to improve the extraction efficiency. In some embodiments, the low-temperature extraction step is performed at 45-60° C. for 50-70 min. In some embodiments, the high-temperature extraction step is performed at 80-90° C. for 50-70 min. Herein, the extraction is to mix the Chrysophyllum cainito with cellulose complex solution, and maintain the mixture at a specific temperature for a specific period of time.

In some embodiments, the Chrysophyllum cainito extract is extracted from the early-harvested fruits of the ice crystal wall-broken Chrysophyllum cainito by taking cellulase complex solution as a solvent, and the cellulase complex solution is prepared by adding 0.1% of cellulase complex in water. In some embodiments, the Chrysophyllum cainito extract is extracted from the early-harvested ice crystal wall-broken fruits of the Chrysophyllum cainito by taking cellulase complex solution as a solvent, performing low-temperature extraction step, and then performing high-temperature extraction step. The cellulase complex solution is prepared by adding 0.1% of cellulase complex in the water.

In some embodiments, the Chrysophyllum cainito extract promotes production of osteoblast by osteocalcin of the subject to achieve the effect of improving bone quality. Osteocalcin is also referred to as Bone Gla Protein (BGP), which is an indicator of osteogenesis rate. Osteocalcin is produced by osteoblast, and the amount of osteocalcin produced reflects osteoblast activity. The amount of osteocalcin produced is recently used as one of a systemic health and anti-aging indicator.

In some embodiments, the Chrysophyllum cainito extract promotes osteocalcin content in blood of the subject to achieve the effect of improving bone quality. The content of osteocalcin in blood has a positive correlation with osteogenesis.

In some embodiments, the Chrysophyllum cainito extract improves bone density (T Score) of the subject to achieve the effect of improving bone quality. Medically, the bone density test results are compared to the optimal bone density of healthy adults at 30 years old to calculate a comparison value, which is referred to as T-Score. That is, when T-Score is 0, it indicates that the bone density is equal to the average value of healthy young people. When T-Score<0, it is indicated as a negative number, and the greater the value of negative number (in units of standard deviation), the lower the bone density.

In some embodiments, the Chrysophyllum cainito extract is used for manufacturing a composition for promoting cartilage differentiation. In some embodiments, the Chrysophyllum cainito extract promotes secretion of glycosaminoglycans (GAGs) by chondrocytes in the subject to achieve the effect of promoting cartilage differentiation. The glycosaminoglycans are natural compounds and can increase synthesis of collagen in the chondrocytes and reduce generation of pro-inflammatory factors, and then cell apoptosis is reduced.

In some embodiments, a manufacturing method of a Chrysophyllum cainito extract is provided and includes: steps of ice crystal wall breaking, low-temperature extraction and high-temperature extraction. Firstly, early-harvested fruits of Chrysophyllum cainito are subjected to standing at −10° C. to 0° C. for 7 d to form wall-broken raw materials (the ice crystal wall breaking step); then, the wall-broken raw materials and water are mixed at 55±5° C. for 60 min to form primary extraction liquid (low-temperature extraction), and 0.1% of cellulase complex is added to the water. Then, the primary extraction liquid is heated to 85±5° C. for 60 min (high-temperature extraction) to form the Chrysophyllum cainito extract.

In some embodiments, a crushing step is further included between thee ice crystal wall breaking step and the low-temperature extraction, and the crushing step is to crush the wall-broken raw materials by a crusher. In some embodiments, the crushing pore size of the crusher is set to be about 30 mm. In some embodiments, the crushing step is to crush water and the wall-broken raw materials together by the crusher. In some embodiments, the crushing step is to crush the water and the wall-broken raw materials together by the crusher and then screen the crushed materials with a screen mesh, and the mesh number of the screen mesh may be 400. In some embodiments, a filtering step is further included after the high-temperature extraction, and primary extract liquid after the high-temperature extraction is filtered by a 400-mesh filter screen to remove fine solids. In some embodiments, a concentration step is further included after the high-temperature extraction; when vacuum concentration is performed at 60° C.±5° C. to reach the Brix (Degrees Brix) of 7.5±0.5 of the primary extract liquid, concentration is stopped, and the Chrysophyllum cainito extract is obtained.

In some embodiments, the Chrysophyllum cainito extract includes at least 30 ppm of myricetin.

In some embodiments, the composition is a food composition, and the composition includes at least a specific amount of the Chrysophyllum cainito extract. In some embodiments, the specific amount of the Chrysophyllum cainito extract is 2 g/d.

In some embodiments, the composition may be a pharmaceutical product. In other words, the pharmaceutical product includes an effective amount of the Chrysophyllum cainito extract. In some embodiments, the effective amount of the Chrysophyllum cainito extract is 2 g/d.

As used herein, the terms “specific use amount” or “effective amount” refer to the amount of a substance required to induce a specified effect in an individual. As will be recognized by those skilled in the art, the specific dosage or effective content will vary depending on the route of administration, use of excipients, and the possibility of co-administration with other substances.

In some embodiments, the aforementioned pharmaceutical product can be manufactured into a dosage form suitable for enteral or oral administration using techniques well known to those skilled in the art. Among them, dosage forms suitable for enteral or oral administration may be, but are not limited to: tablets, troche, lozenges, pills, capsules, dispersible powders or granules, solutions, suspension, emulsion, syrup, elixir, slurry, or the like.

In some embodiments, the aforementioned pharmaceutical product can be manufactured into a dosage form suitable for parenterally or topically administration using techniques well known to those skilled in the art. Among them, dosage forms suitable for parenteral or topical administration may be, but are not limited to, injections, sterile powders, external manufactures, and the like. In some embodiments, the pharmaceutical product may be administered by parenteral routes selected from the group consisting of: subcutaneous injection, intraepidermal injection, intradermal injection, or intralesional injection.

In some embodiments, the pharmaceutical product may further include pharmaceutically acceptable carriers that are widely used in pharmaceutical manufacturing technology. For example, a pharmaceutically acceptable carrier may include one or more of the following reagents: solvents, buffers, emulsifiers, suspending agents, decomposers, disintegrating agents, dispersing agents, binding agents, excipients, stabilizing agents, chelating agents, diluents, gelling agents, preservatives, wetting agents, lubricants, absorption delaying agents, liposomes and the like. The selection and quantities of these reagents are within the professionalism and routine skills of those skilled in the art.

In some embodiments, the pharmaceutically acceptable carrier may further include solvents selected from the group consisting of: water, normal saline, phosphate buffered saline (PBS), aqueous solution including alcohol.

In some embodiments, the aforementioned composition can be an edible composition for non-medical purposes, and the edible composition at least includes a specific amount of Chrysophyllum cainito extract. In some embodiments, the edible composition can be made into a food product or can be a food additive. That is, the Chrysophyllum cainito extract of any of the foregoing embodiments can be added during the manufacture of food materials by conventional methods to manufacture food products, or the Chrysophyllum cainito extract of any of the aforementioned embodiments can be added directly during the production of food products. Therefore, the food product may be a product formulated with edible materials for ingestion by humans or animals. In some embodiments, the edible composition may further include a food ontology, or a food ontology and a food additive.

In some embodiments, the food product may be, but is not limited to: beverages, fermented foods, bakery products, health foods for non-medical purposes, or dietary supplements for non-medical purposes, etc.

### Embodiment I: Manufacturing of Test Samples

### 1-1. Manufacturing of a Chrysophyllum cainito Extract in an Experimental Group

Intact fresh early-harvested fruits (green skin) of Chrysophyllum cainito produced in Taiwan, China were harvested, and the intact fruits included peels, seeds and fleshes; the intact fruits were subjected to standing at −10° C. for 7 days to form wall-broken raw materials (ice crystal wall-breaking step). The wall-broken raw materials were added into cellulase complex solution, and the wall-broken raw materials and the cellulase complex solution were mixed in a weight ratio of 1:10. Then, the wall-broken raw materials and the cellulase complex solution were crushed and mixed (in this process, smashing and mixing were performed by an SAMPO KJ-SD15G device, and the coarse crushing pore diameter was 30 mm), and the cellulase complex solution is prepared by adding 0.1% of cellulase complex in water. In this process, the cellulase complex was purchased from novozymes and included 59.9% of water, 23% of sucrose, 10% of sodium chloride, 7% of endo-1,3(4)-β-glucanase, and 0.1% of potassium sorbate.

The wall-broken raw materials and cellulase complex solution heated to reach 55±5° C. and mixed for 60 min to obtain primary extract liquid (low-temperature extraction).

Then, the primary extract liquid was heated to 85° C., and maintained for 1 hour to obtain secondary extract liquid. Then, solids were filtered from the secondary extract liquid through a 400-mesh screen mesh, and then vacuum concentration was performed; and when the vacuum concentration was performed at 60±5° C. to reach the Degrees Brix of 7.5±0.5 of the solution, the concentration was stopped to obtain the Chrysophyllum cainito extract. In this process, the brand/model of a concentrator was BUCHI-Rotavapor R-100.

### 1-2. Manufacturing of a Chrysophyllum cainito Extract in a Control Group

Intact fresh ripe fruits (pulp skin) of Chrysophyllum cainito produced in Taiwan, China were harvested, and the intact fruits included peels, seeds and fleshes; the intact fruits were subjected to standing at −10° C. for 7 days to form wall-broken raw materials (ice crystal wall-breaking step). The wall-broken raw materials were added into a cellulase complex solution, and the wall-broken raw materials and the cellulase complex solution were mixed in a weight ratio of 1:10. Then, the wall-broken raw materials and the cellulase complex solution were crushed and mixed (in this process, smashing and mixing were performed by an SAMPO KJ-SD15G device, and the coarse crushing pore diameter was 30 mm), and the cellulase complex solution is prepared by adding 0.1% of cellulase complex in water. In this process, the cellulase complex was purchased from novozymes and included 59.9% of water, 23% of sucrose, 10% of sodium chloride, 7% of endo-1,3(4)-β-glucanase, and 0.1% of potassium sorbate.

The wall-broken raw materials and cellulase complex solution heated to reach 55±5° C. and mixed for 60 min to obtain primary extract liquid (low-temperature extraction).

Then, the primary extract liquid was heated to 85° C., and maintained for 1 hour to obtain secondary extract liquid. Then, solids were filtered from the secondary extract liquid through a 400-mesh screen mesh, and then vacuum concentration was performed; and when the vacuum concentration was performed at 60±5° C. to reach the Degrees Brix of 7.5±0.5 of the solution, the concentration was stopped to obtain the ripe fruit extract. In this process, the brand/model of a concentrator was BUCHI-Rotavapor R-100.

### Embodiment II: Test of Promoting Osteocalcin Secretion

### 2-1. Materials and Instruments

Experimental cell line: Murine bone marrow stromal cells (hereinafter referred to as OP9 cells) were used, and the OP9 cells were purchased from OP9 cell lines (ATCC CRL-2749™) from the American Type Culture Collection (ATCC®).

Cell culture medium: It included 90% of MEMAM (Minimum Essential Medium Alpha Medium, purchased from Gibco, product number: Cat. 12000-022), 20% of Fetal Bovine Serum (purchased from Gibco, Cat. 10437-028), and 1% of Penicillin-streptomycin (purchased from Gibco, Cat. 15240-062).

Differentiation culture medium: It included 90% of DMEM (Dulbecco's modified Eagle's medium, purchased from Gibco, Cat. 12100-038) added with additional ingredients to make it include 10% of FBS (fetal bovine Serum, purchased from Gibco, 10438-026) and 1% of penicillin-streptomycin (purchased from Gibco, Cat. 15140122), 50 μM of ascorbic acid (purchased from Sigma), 10⁻⁷ M of dexamethasome (purchased from Sigma), and 10 mM of β-glycerol (purchased from Sigma).

Dulbecco's Phosphate-Buffered Saline (DPBS solution): Purchased from Gibco, product number: 14200-75.

ELISA Kit for Osteocalcin (OC): Brand: USCN; model: SEA471Mu.

Flow cytometer: Purchased from BD Pharmingen Company, model: BDTM Accuri C6 Plus.

### 2-2. Test Steps

OP9 cells were inoculated into a 24-well culture disc including 2 mL of cell culture medium in each well according to the density of 2×10⁴ in each well, and cultured in a CO² incubator for 24 h. Then, the cell culture medium was replaced with a fresh differentiation culture medium.

The culturing was continued for 7 d, and the fresh differentiation culture medium was replaced every 3 days.

The OP9 cells were divided into a blank group, an experimental group and a control group, and continuously cultured for 7-10 days, and the fresh differentiation culture medium was replaced every 3 days. 0.03125 mg/mL of the Chrysophyllum cainito extract manufactured in Embodiment I was added into the differentiation culture medium in the experimental group. No test sample was added into the blank group, and the blank group was a pure differentiation culture medium. 0.03125 mg/mL of the ripe fruit extract manufactured in Embodiment I was added into the differentiation culture medium in the control group.

The cell pattern was observed to determine that the OP9 cells were differentiated into hardbone cells.

Supernate was collected from each well of the culture disc into a micro centrifuge tube for subsequent detection by the ELISA Kit for osteocalcin. In this process, ELISA analysis was performed by the ELISA Kit for osteocalcin according to an operation book provided by the manufacturer.

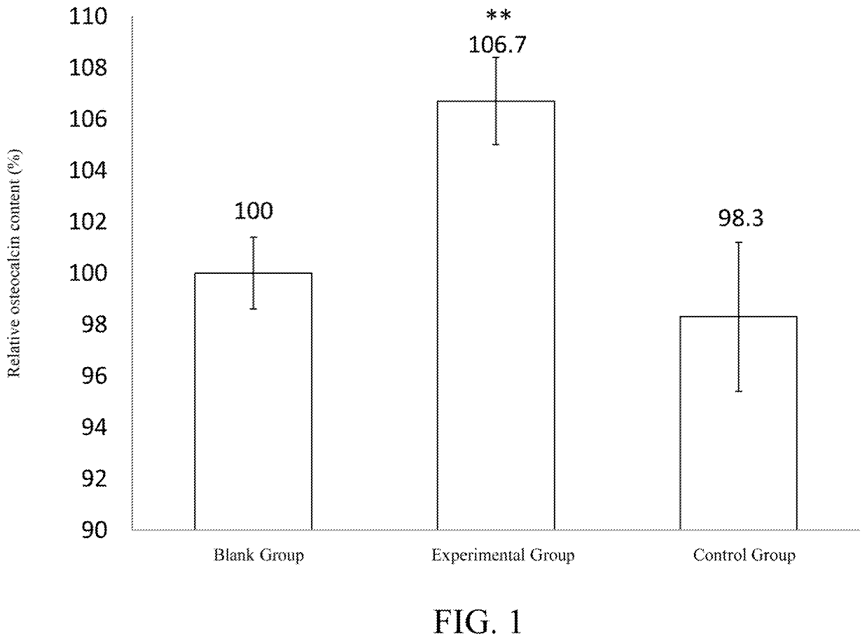

The content of osteocalcin in the supernatant to be detected in each well was measured by the flow cytometer to promote osteocalcin secretion analysis. The operation was repeated three times for each group, so the triple repeated experimental results of each group were averaged to obtain an average value; then the osteocalcin secretion average value of the blank group was taken as 100%, and the average value of the experimental group and the control group was converted into the relative osteocalcin secretion amount, as shown in FIG. 1.

### 2-3. Test Results

The obtained results are subjected to student t-test with Excel software to determine whether there is a statistically significant difference between the two sample groups, as shown in FIG. 1, “*” represents the p value less than 0.05, and “**” represents the p value less than 0.01, and “***” represents the p value less than 0.001. The more “*” there are, the more statistically significant the difference is compared to the blank group.

As shown in FIG. 1, the relative osteocalcin secretion of the experimental group was 106.7%, so the effect of promoting osteocalcin secretion of bone cells was achieved. In contrast, compared with the blank group, osteocalcin secretion of the bone cells of the control group was reduced to 98.3%. It indicates that the Chrysophyllum cainito extract promoted production of osteocalcin secretion of bone cells, but the ripe fruit extract did not have this effect.

### Embodiment III: Test of Promoting Glycosaminoglycan Secretion

The murine cartilage precursor cells can be differentiated into chondrocytes. According to this test, the expression state of an Alcian blue stain and ELISA reader quantification were observed through a microscope, the degree of differentiation of the murine cartilage precursor cells into the chondrocytes was analyzed, and then whether the tested sample has the capability of promoting cartilage differentiation or not was evaluated. The glycosaminoglycan was stained with the Alcian blue stain, and the differentiation state of the cells could be observed by observing the number of the glycosaminoglycans.

### 3-1. Materials and Instruments

Cell line: Murine cartilage precursor cells ATDC5 (purchased from Sigma, model: 99072806).

ATDC5 culture medium: DMEM (Dulbecco's modified Eagle's medium, purchased from Gibco, Cat. 12100-038) basic culture medium and Ham's F-12 basic medium were mixed in a ratio of 1:1, and additional ingredients were added to make it include 5% of FBS (fetal bovine Serum, purchased from Gibco, 10438-026), 2 mM L of Glutamine (purchased from Gibco), 1% of penicillin/streptomycin (purchased from Gibco).

Differentiation culture medium: DMEM (Dulbecco's modified Eagle's medium, purchased from Gibco, Cat. 12100-038) basic culture medium and Ham's F-12 basic medium were mixed in a ratio of 1:1, and additional ingredients were added to make it include 5% of FBS (fetal bovine Serum, purchased from Gibco, 10438-026), 2 mM of Glutamine (purchased from Gibco), 1% of penicillin/streptomycin (purchased from Gibco) and 1% of Insulin-transferrin-Selenium.

Alcian blue: Purchased from Sigma.

Dulbecco's Phosphate-Buffered Saline (DPBS solution): Purchased from Gibco, product number: 14200-75.

Formaldehyde: Purchased from Gibco, product number: TG1794-4-0000-72NI.

HCl: Concentration: 0.1 M.

Trypsin: 10× Trypsin-EDTA (purchased from Gibco, product number: 15400-054).

### 3-2. Test Flow

The murine cartilage precursor cells were inoculated into a 6-well culture disc including 2 mL of ATDC5 culture medium in each well according to a density of 1×10⁵ cells in each well; the culture disc was placed for culturing at 37° C. under 5% of CO₂; the culturing was continued until the disc was full of cells; and a fresh ATDC5 culture medium was replaced every 3 days during culturing.

The murine cartilage precursor cells were divided into a blank group and an experimental group, and continuously cultured for 35 days, and a fresh differentiation culture medium was replaced every 3 days. 0.03125 mg/mL of the Chrysophyllum cainito extract manufactured in Embodiment I was added into the differentiation culture medium in the experimental group. No test sample was added into the blank group, and the blank group was a pure differentiation culture medium.

Then, Alcian blue stain staining treatment was performed, and the treatment included the following steps: removing the differentiation culture medium, and rinsing for 3 times with 1×PBS; adding 0.5 mL of 10% formaldehyde, and fixing at room temperature for 30 min; removing the formaldehyde, and rinsing for 3 times with 1×PBS; manufacturing 1% of Alcian blue stain (pH=2.5) by HCl; and adding 0.5 mL of Alcian blue stain into each well, and staining at room temperature overnight.

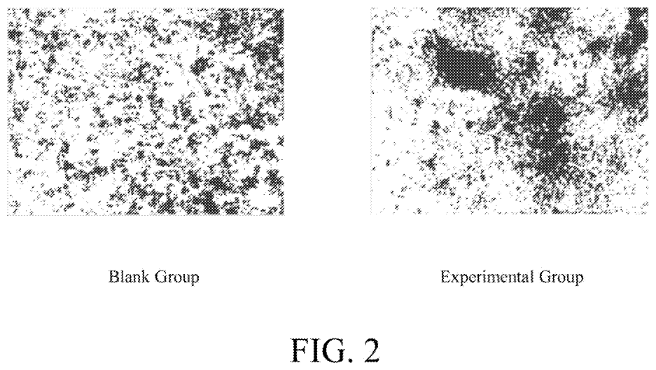

Finally, observing and quantifying were performed, and the treatment included: rinsing for 3 times with 1×PBS; observing a blue signal presented by the Alcian blue stain staining glycosaminoglycan through the microscope; taking a picture through the microscope; performing soft color quantitative analysis by Fiji software; and analyzing the blue signal of the Alcian blue stain; and the result reproduced in black-and-white was shown in FIG. 2.

### 3-3. Test Results

As shown in FIG. 2, the black color was relatively spare in the blank group, while the black color was quite dense in the experimental group; in other words, more glycosaminoglycans were observed in the experimental group. Based on this, the test results showed that early-harvested fruit of Chrysophyllum cainito could promote the differentiation of precursor cells into chondrocytes and promote the chondrocytes to generate more glycosaminoglycans to protect joints.

### Embodiment IV: Human Experiment

Sample: Chrysophyllum cainito extract manufactured in Embodiment I.

Subjects: 9 subjects. The subjects were women aged over 35 and under 70. Generally, the medical reference value of osteocalcin is 11.0 ng/mL to 43.0 ng/mL for women before menopause and 15.0 ng/mL to 46.0 ng/mL for postmenopausal women. Herein, in the dynamic balance of bones, if the osteogenesis rate is too slow and lower than the resorption rate, it will easily lead to osteoporosis for a long time. As far as osteocalcin, the low concentration of osteocalcin may mean that the osteogenesis rate is too slow, and the high concentration means that the rate of bone replacement is too fast, both of which are signs of osteoporosis. According to the classification of osteoporosis by the World Health Organization (WHO), the T score from +1 to −1 represents normal, the T score from −1 to −2.5 represents osteopenia, and the T score from −2.5 or less represents osteoporosis.

### 4-1. Test Items

Items: Concentration of osteocalcin in blood, and bone density.

Blood was used as the sample for testing concentration of osteocalcin, and the commissioned testing unit was Harvard Health Clinic.

The bone density was measured by a dual energy X-ray absorptiometry (DXA), which measured the bone density of the lumbar spine and converted it into a T score performance. Herein, the average bone density of the first to fourth lumbar vertebrae was used for conversion.

### 4-2. Test Flow

9 subjects were asked to take 2 g of Chrysophyllum cainito extract every day and continue to take it for twelve weeks. Before starting the intake (marked as the 0^{th} week in the figure), after eight weeks of intake (marked as the 8^{th} week in the figure), and after twelve weeks of intake (marked as the 12^{th} week in the figure), blood was correspondingly collected and measured with the above device.

### 4-3. Test Results

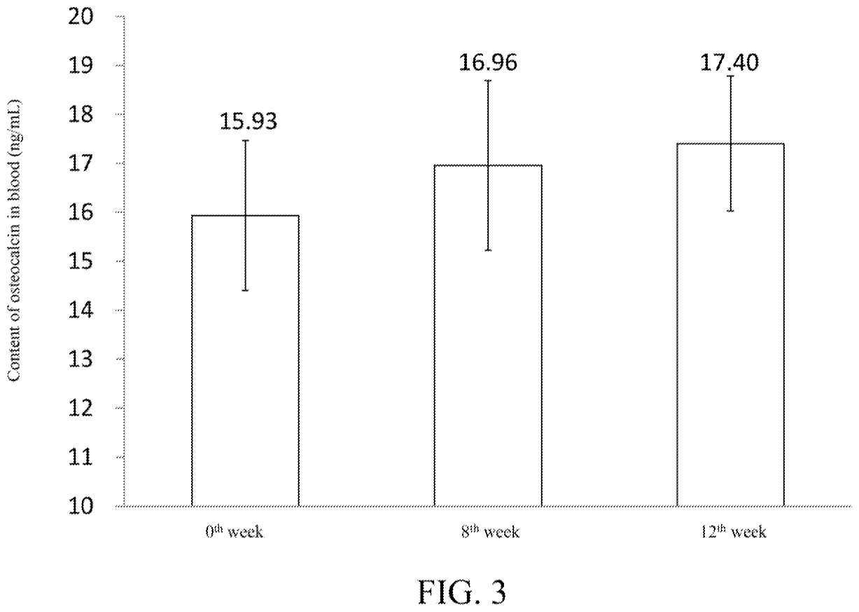

As shown in FIG. 3. The average content of osteocalcin in blood at the 0^{th} week was 15.93 ng/mL. After 8 weeks of daily intake of 2 g of Chrysophyllum cainito extract, the average content of osteocalcin in blood of the 9 subjects increased to 16.96 ng/mL; and after 12 weeks of daily intake of 2 g of Chrysophyllum cainito extract, the average content of osteocalcin in blood of the 9 subjects increased to 17.40 ng/mL. That is, after taking Chrysophyllum cainito extract for 8 weeks, the content of osteocalcin in blood increased by 6.5%; and after 12 weeks of continuous use, the content of osteocalcin in blood increased by 9.2%.

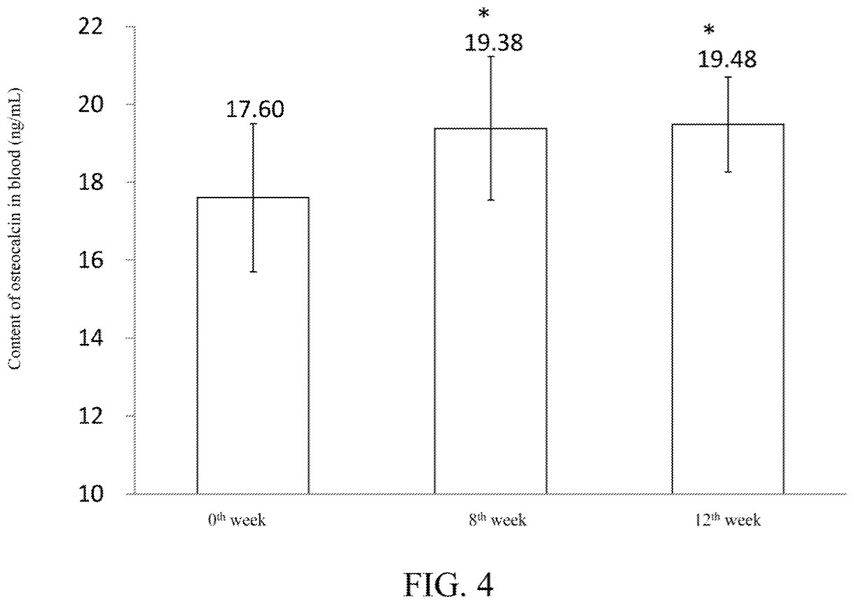

As shown in FIG. 4. The conditions of 6 postmenopausal subjects among the subjects were separately observed. It could be seen that the average content of osteocalcin in blood of these 6 subjects at the 0^{th} week was 17.60 ng/mL. After 8 weeks of daily intake of 2 g of Chrysophyllum cainito extract, the average content of osteocalcin in blood of the 6 subjects increased to 19.38 ng/mL; and after 12 weeks of daily intake of 2 g of Chrysophyllum cainito extract, the average content of osteocalcin in blood of the 6 subjects increased to 19.48 ng/mL. That is, after taking Chrysophyllum cainito extract for 8 weeks, the content of osteocalcin in blood increased by 10.1%; and after 12 weeks of continuous use, the content of osteocalcin in blood increased by 10.7%.

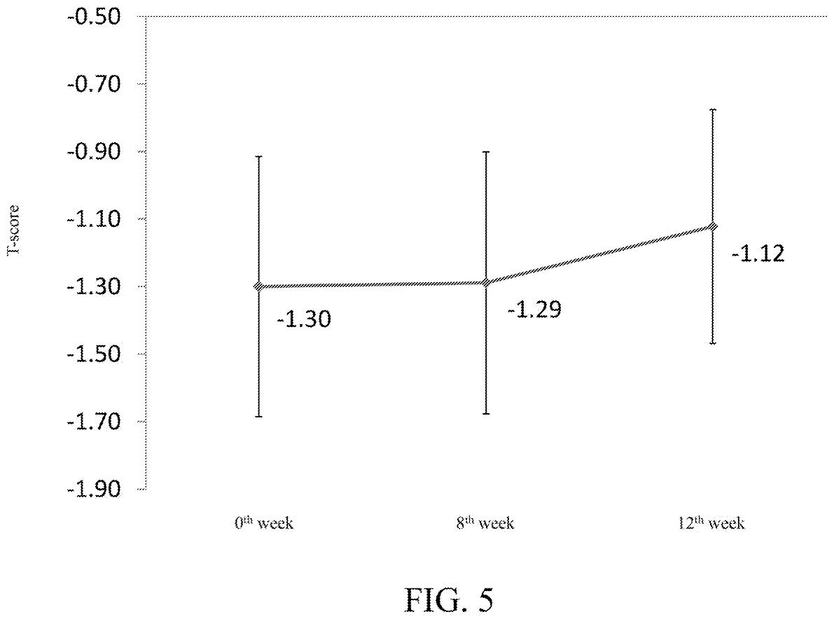

As shown in FIG. 5. The average T score at the 0^{th} week was −1.30. After 8 weeks of daily intake of 2 g of Chrysophyllum cainito extract, the average T score of the 9 subjects increased to −1.29; and after 12 weeks of daily intake of 2 g of Chrysophyllum cainito extract, the average T score of the 9 subjects increased to −1.12. That is, after 12 weeks of taking the Chrysophyllum cainito extract, the bone density increased by 13.9%.

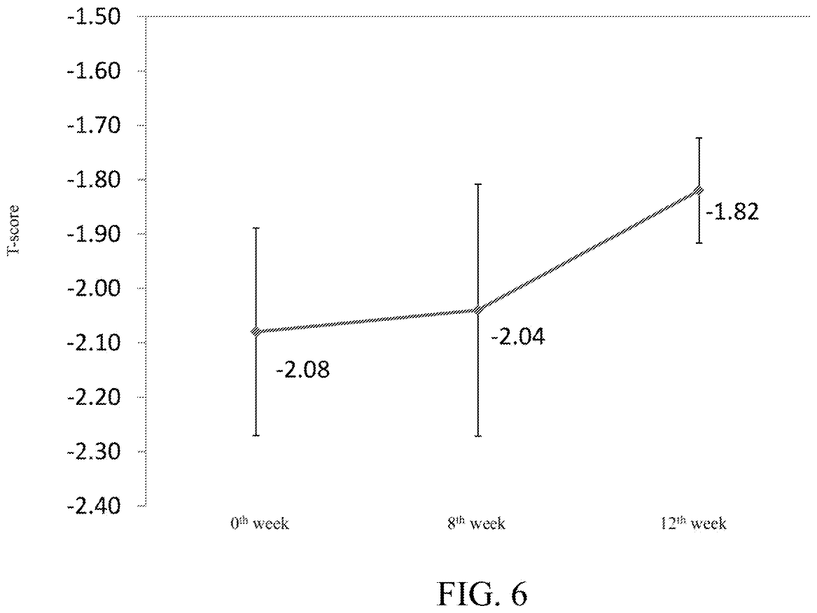

As shown in FIG. 6. The conditions of 6 postmenopausal subjects among the subjects were separately observed. It could be seen that the average T score of these 6 subjects at the 0^{th} week was −2.08; after 8 weeks of daily intake of 2 g of Chrysophyllum cainito extract, the average T score of the 6 subjects improved to −2.04; and after 12 weeks of daily intake of 2 g of Chrysophyllum cainito extract, the average T score of the 6 subjects improved to −1.82. That is, after 12 weeks of taking the Chrysophyllum cainito extract, the bone density increased by 12.5%.

### Embodiment V: Fingerprint HPLC Test of Chrysophyllum cainito Extract

In this process, a High Performance Liquid Chromatography (HPLC) was used for quantitatively and qualitatively analyzing bioactive substances in the Chrysophyllum cainito extract manufactured in Embodiment I.

Solvents used in this test were methanol and water, and 0.1% formic acid was added to the methanol and the water respectively; the flow rate was set to be 1 ml/min; the elution conditions were set as follows: the ratio of the methanol to the water was 2:98 in 0 min; the ratio of the methanol to the water was 2:98 in 10 min; the ratio of the methanol to the water was 70:30 in 40 min; the ratio of the methanol to the water was 100:0 in 50 min; and the ratio of the methanol to the water was 100:0 in 60 min.

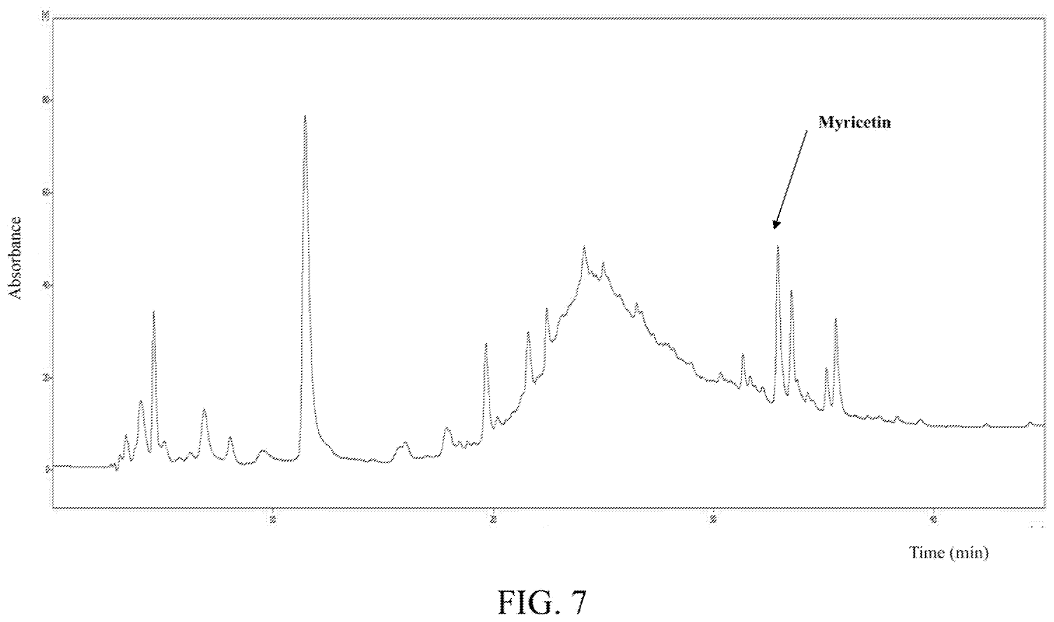

As shown in FIG. 7. The peak of the bioactive substance resolved in about 33 min was confirmed to be myricetin after analysis and comparison. Meanwhile, the HPLC quantitative analysis result showed that the myricetin content in the Chrysophyllum cainito extract was 30.2 ppm.

### Embodiment VI: Test of Myricetin Promoting Osteocalcin Secretion

### 6-1. Materials and Instruments

Experimental cell line: Murine bone marrow stromal cells (hereinafter referred to as OP9 cells) were used, and the OP9 cells were purchased from OP9 cell lines (ATCC CRL-2749™) from the American Type Culture Collection (ATCC®).

Cell culture medium: It included 90% of MEMAM (Minimum Essential Medium Alpha Medium, purchased from Gibco, product number: Cat. 12000-022), 20% of Fetal Bovine Serum (purchased from Gibco, Cat. 10437-028), and 1% of Penicillin-streptomycin (purchased from Gibco, Cat. 15240-062).

Differentiation culture medium: It included 90% of DMEM (Dulbecco's modified Eagle's medium, purchased from Gibco, Cat. 12100-038) added with additional ingredients to make it include 10% of FBS (fetal bovine Serum, purchased from Gibco, 10438-026) and 1% of penicillin-streptomycin (purchased from Gibco, Cat. 15140122), 50 μM of ascorbic acid (purchased from Sigma), 10⁻⁷ M of dexamethasome (purchased from Sigma), and 10 mM of β-glycerol (purchased from Sigma).

Dulbecco's Phosphate-Buffered Saline (DPBS solution): Purchased from Gibco, product number: 14200-75.

ELISA Kit for Osteocalcin (OC): Brand: USCN; model: SEA471Mu.

Flow cytometer: Purchased from BD Pharmingen Company, model: BDTM Accuri C6 Plus.

### 6-2. Test Steps

OP9 cells were inoculated into a 24-well culture disc including 2 mL of cell culture medium in each well according to the density of 2×10⁴ in each well, and cultured in a CO² incubator for 24 h. Then, the cell culture medium was replaced with a fresh differentiation culture medium.

The culturing was continued for 7 days, and the fresh differentiation culture medium was replaced every 3 days.

The cell pattern was observed; after determining that the OP9 cells were differentiated into hardbone cells, the hardbone cells were divided into a blank group and an experimental group, and continuously cultured for 7 days, and the fresh differentiation culture medium was replaced every 3 days. 0.03125 mg/mL of myricetin was added into the differentiation culture medium of the experimental group. No test sample was added into the blank group, and the blank group was a pure differentiation culture medium.

Supernatant was collected from each well of the culture disc into a micro centrifuge tube for subsequent detection by the ELISA Kit for osteocalcin. In this process, ELISA analysis was performed by the ELISA Kit for osteocalcin according to an operation book provided by an original factory.

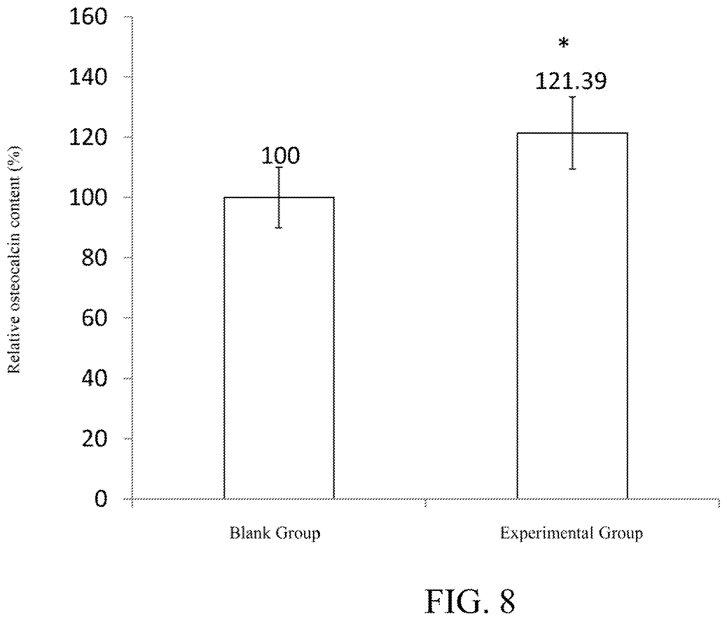

The content of osteocalcin in the supernatant to be detected in each well was measured by the flow cytometer to promote osteocalcin secretion analysis. The operation was repeated three times for each group, so triple repeated experimental results of each group were averaged to obtain an average value; then the osteocalcin secretion average value of the blank group was taken as 100%, and the average value of the experimental group was converted into the relative osteocalcin secretion amount, as shown in FIG. 8.

### 6-3. Test Results

The obtained results were subjected to student t-test with Excel software to determine whether there is a statistically significant difference between the two sample groups, as shown in FIG. 8, “*” represents the p value less than 0.05, and “**” represents the p value less than 0.01, and “***” represents the p value less than 0.001. The more “*” there are, the more statistically significant the difference is compared to the blank group.

As shown in FIG. 8, the relative osteocalcin secretion of the experimental group was 121.39%, so the effect of promoting the osteocalcin secretion of bone cells was achieved. It indicates that the bioactive substance, myricetin, in the Chrysophyllum cainito extract promoted osteocalcin secretion of the bone cells.

In conclusion, the Chrysophyllum cainito extract in any embodiment of the present disclosure can be used for manufacturing the composition for improving bone quality and promoting cartilage differentiation. In some embodiments, the Chrysophyllum cainito extract has at least one of the following effects of: promoting production of osteocalcin by osteoblasts, promoting osteocalcin content in blood, improving bone density of the lumbar vertebra, and promoting secretion of glycosaminoglycan by chondrocytes.

Although the present disclosure has been described in considerable detail with reference to certain preferred embodiments thereof, the disclosure is not for limiting the scope of the invention. Persons having ordinary skill in the art may make various modifications and changes without departing from the scope and spirit of the disclosure. Therefore, the scope of the appended claims should not be limited to the description of the preferred embodiments described above.

What is claimed is:

1. A method for improving bone quality of a subject in need thereof, comprising: administering to the subject an effective amount of Chrysophyllum cainito extract, wherein the Chrysophyllum cainito extract is obtained by extracting early-harvested fruit of Chrysophyllum cainito by a cellulase complex solution, and the cellulase complex solution is prepared by adding 0.1% of cellulase complex in water.

2. The method according to claim 1, wherein a weight ratio of the cellulase complex solution to the early-harvested fruits of Chrysophyllum cainito is (5-10):1.

3. The method according to claim 1, wherein the extraction of early-harvested fruits of Chrysophyllum cainito comprises steps of: ice crystal wall breaking, low-temperature extraction, and high-temperature extraction; wherein, the ice crystal wall breaking step is performed by letting the early-harvested fruit stand at −10° C. to 0° C. for 7 days, the low-temperature extraction step is performed at 45-60° C. for 50-70 min, and the high-temperature extraction step is performed at 85±5° C. for 50-70 min.

4. The method according to claim 1, wherein the Chrysophyllum cainito extract promotes production of osteocalcin by osteoblasts of the subject to achieve the effect of improving bone quality.

5. The method according to claim 4, wherein the Chrysophyllum cainito extract promotes osteocalcin content in blood of the subject to achieve the effect of improving bone quality.

6. The method according to claim 1, wherein the Chrysophyllum cainito extract improves bone density of lumbar vertebra of the subject to achieve the effect of improving bone quality.

7. A method for promoting cartilage differentiation in a subject in need thereof, comprising: administering to the subject an effective amount of Chrysophyllum cainito extract, wherein the Chrysophyllum cainito extract is obtained by extracting early-harvested fruit of Chrysophyllum cainito by a cellulase complex solution, and the cellulase complex solution is prepared by adding 0.1% of cellulase complex in water.

8. The method according to claim 7, wherein the Chrysophyllum cainito extract promotes secretion of glycosaminoglycans (GAGs) by chondrocytes in the subject to achieve the effect of promoting cartilage differentiation.

9. A manufacturing method of Chrysophyllum cainito extract, comprising a step of: extracting early-harvested fruit of Chrysophyllum cainito by a cellulase complex solution prepared by adding 0.1% of cellulase complex in water,
wherein the extraction comprises steps of: ice crystal wall breaking, low-temperature extraction, and high-temperature extraction; wherein, the ice crystal wall breaking step is performed by letting the early-harvested fruit stand at −10° C. to 0° C. for 7 days, the low-temperature extraction is performed at 45-60° C. for 50-70 min, and the high-temperature extraction is performed at 85±5° C. for 50-70 min.

10. The method according to claim 9, wherein the Chrysophyllum cainito extract comprises at least 30 ppm of myricetin.
